(19) United States
(12) Patent Application Publication
(10) Pub. No.: US 2015/0034097 A1
(43) Pub. Date: Feb. 5, 2015

(54) DEVICE AND METHOD FOR RESTRAINING FOOD IN TAKE

(71) Applicant: NOBESITY LTD., HAIFA (IL)

(72) Inventors: Erella Pines, Pardes Hana (IL); Jacob Dagan, Tel Aviv (IL); Yoram Pony, Moshav Rarnat Raziel (IL)

(21) Appl. No.: 14/385,355

(22) PCT Filed: Mar. 14, 2013
(86) PCT No.: PCT/IB2013/000807

§ 371 Date: Sep. 15, 2014

Related U.S. Application Data
(60) Provisional application No. 61/611,351, filed on Mar. 15, 2012.

(51) Int. Cl. A61F 5/00

(52) U.S. Cl. CPC A61F 5/0006
USPC 128/859

(57) ABSTRACT

An intra-oral device and a method of using the device to assist overweight persons in treating obesity by reducing and/or limiting the mouth opening and/or slowing down the rate of chewing. Such a device is handled by the user with or without the need for professional help, by increasing the awareness of the user about the need to reduce the amount of food intake in order to lose weight. The intra-oral device is intended to train the user to reduce the mouth opening. The intra-oral device is a restraining device which is anchored in the upper and lower jaws. The restraining device is an elastic element which is inserted between two respective inter-proximal spaces (IPSs) of the upper and the lower jaws of the user. The device reduces and/or limits the mouth opening and/or slows down the rate of chewing. The device is disposable, intended for a single use, and is removable after use. The device can be disengaged in case of a need to open the mouth above the limits of the device.

### CROSS REFERENCE TO RELATED APPLICATIONS

The present application claims priority to U.S. Provisional Application Ser. No. 61/611,351 filed Mar. 15, 2012 and is related to U.S. patent application Ser. No. 12/808,364, filed Jun. 15, 2010, which issued as U.S. Pat. No. 8,375,954 on Feb. 19, 2013, the disclosures of which are expressly incorporated by reference herein in their entirety.

### BACKGROUND OF THE INVENTION

1. Field of the Invention

The present invention relates to intra-oral devices for treating overweight persons for obesity by altering eating habits, and explicitly by alerting the user to reduce and/or limit the mouth opening and/or slow down the rate of chewing. More specifically, the present invention relates to such devices handled by the user with or without the need for professional help. Furthermore, the present invention relates to a method and the use of such a device.

2. Discussion of Background Information

Overweight people who suffer from obesity have some of the most common health and social problems. Millions of people suffer from being overweight. Obesity is considered to be a main cause for diseases and early death. In developed countries, obesity is becoming a national public health problem. Unfortunately, no single treatment is available for treating the condition satisfactorily. The International Health Organization refers to obesity as an epidemic, and has formed a special task force to treat it as one of the biggest risks threatening human health.

Obesity occurs when there is an excess of energy consumption in relation to energy release. The cause for people being overweight differs from patient to patient, and it is commonly believed to be associated with several genetic, psychological, social, behavioral and environmental factors.

People who suffer from being overweight tend to belong to risk groups prone to stress, obesity-associated disorders, heart attacks, diabetes, strokes, gall bladder diseases, death while sleeping, respiratory problems and depression. The goals of obesity treatment are to reduce the risks of developing related diseases or the aggravation of existing ones, to lengthen life expectancy, and to improve the way and quality of life. Treating overweight persons usually involves diets, psychological treatments, psychiatric treatments, use of drugs, alternative medicine, physical activity and surgical intervention. The challenge of treating moderate obesity is to find an approach which is effective in maintaining reduced weight because after weight reduction, people are often prone to regain the weight. This is the point at which behavioral therapy is effective.

In recent years, researchers have reached the conclusion that the most successful approach for the treatment of obesity is a multi-disciplinary one. To succeed in the long term, a combination of behavioral therapy, e.g. changing eating habits, and other treatments is required. According to the National Institutes of Health's Guide to Behavior Change, slowing the rate of eating may allow satiety, i.e. fullness, signals to begin to be transmitted by the end of the meal. Recent studies show that dietary behavioral modifications and exercise are the most efficient methods for managing body weight. The present invention is related to a device configured to aid weight loss by controlling the amount of food a person bites and the rate of mastication, i.e. reducing and/or limiting the mouth opening and/or slowing down the rate of chewing.

Slow and thorough chewing enables better and more complete absorption of the food in the stomach, and decreases the hunger feeling. To the contrary, fast eating promotes incomplete food absorption and encourages the eater to consume more in order to feel full. Another way to eat less and feel full is to give the brain time to get the message that the stomach is full. Because it takes 15 minutes or more to get the signal to the brain, eating slowly, as well as chewing and grinding slowly, are effective in this respect.

Oral devices and more specifically dental appliances have been used to control weight by slowing the rate of food mastication. The body is allowed time to respond to the ingestion of food with a sensation of satiety. The user is supposed to feel full after consuming a reduced amount of food. Therefore, the patient eats less and as a result loses weight.

A number of dental appliances have been described as aiding a user in achieving weight loss through the restriction of mandible movements.

For example, U.S. Pat. No. 4,471,771 discloses an oral weight control apparatus. The apparatus includes a guard, net, or other sieve-like blocking means secured in the mouth of the user. Liquids and finely ground material inside the mouth may freely pass through the guard or sieve but solid foods may not. In the preferred embodiment, the blocking means functions as a one-way valve, blocking solid foods from entering the stomach through the mouth.

U.S. Pat. No. 4,825,881 discloses an apparatus for inhibiting the intake of food. The apparatus comprises first and second spaced adhesive strips applied above and below the upper and lower lips of the user. These strips are interconnected by wires which provide relatively normal movement of the mouth to permit normal speech, while at the same time inhibiting, but not totally preventing, the intake of solids and liquids.

U.S. Pat. No. 6,138,679 discloses a mandible restraint that includes a pain-inducing device. This pain-inducing device may be a bar-shaped element with a thickened portion that is intended to press against the gums or jaws of the patient if the patient attempts to open his mouth beyond a threshold position. Alternatively, or additionally, the pain-inducing device delivers a painful electric shock to the gums or teeth of the patient.

US Published Application No. 2003/075186 discloses a device for the teeth of a user by engaging a plurality of individual teeth of the mandible with the opposite respective teeth of the maxilla. The affixation is carried out separately for each pair of upper and lower teeth by applying a resin to anchor an elastic element to the teeth.

In US Published Patent Application No. 2003/059737, four frames are secured to the upper and lower right and left dental arches and are connected to magnets on each frame.

Spanish (ES) Patent No. 2164570 describes a closing system for teeth. It consists of rings that are fixed to the teeth and a chain that will pass through the rings to close the teeth.

U.S. Pat. No. 4,218,611 discloses a method and apparatus for controlling a person's eating behavior by using a counter on the table next to the food being eaten. The counter displays the number of bites to be taken at each meal, displays the actual number of bites while the bites are being taken during a meal, provides a cadence signal from which the person can pace his chewing rate, determines the size of the bite taken and the time between bites, and provides an exercise between bites which forces the person to break the eating chain by pushing a button to increment the counter.

Also, there is a device known as “Jaws”. This device joins the upper and lower jaws by a metal wire which is applied by a dentist. However, this device is very dangerous and is not allowed to be used any more.

Today it is obvious that losing weight concerns firstly changing eating habits, done under guidance. None of the above patents provides a satisfactory solution for the need to lose weight.

Moreover, in order to insert a restraining device, in some cases, a helping instrument is required. A number of instruments, such as applicators, have been described as helping to insert certain devices into the oral cavity but most of them are used by the dentist, usually an orthodontist, to fix the devices on orthodontic appliances or are applicators that help the person, usually an orthodontic patient, to put orthodontic devices in their proper places.

U.S. Pat. No. 5,575,643 describes an orthodontic tool wherein an elastic band can be stretched and affixed onto the hooks of an orthodontic apparatus already fitted within the patient's mouth.

U.S. Pat. No. 4,127,940 describes a dentist's instrument used to facilitate the application of an elastic arch wire-retaining band to an arch wire-supporting bracket.

U.S. Pat. No. 4,472,137 describes an orthodontic instrument which is provided for attaching elastic retainer rings or bands to the brackets already affixed to a patient's teeth.

U.S. Pat. No. 4,512,739 describes an orthodontic instrument which is intended for use by patients to help them put on elastic bands and to remove the elastic bands from orthodontic hooks that are cemented on orthodontic brackets.

U.S. Pat. No. 3,475,818 describes an applicator for the placement of elastic bands on orthodontic appliances.

None of the above patents gives a solution as satisfactory as the restraining device according to the present invention.

### SUMMARY OF THE INVENTION

The present invention includes a restraining device which is anchored in the upper and lower jaws of a user. The device has an elastic element which is inserted between two respective inter-proximal spaces (IPSs) of the upper and lower jaws, thus increasing awareness of a user. The device causes the user to reduce and/or limit the mouth opening and/or to slow down the rate of chewing. The elastic element may be referred to as an elastic band. The restraining device, i.e. the elastic element, may be inserted into the mouth opening of the user via a helping instrument, e.g. an applicator.

The restraining device may include at least one inter-dental connector, at least one slow-down chewing ring, and at least one stopper, wherein the connector, the ring and the stopper are connected together.

The elastic element may be manufactured from a strong material which cannot be torn while being inserted into the mouth opening and a flexible material which enables opening and closing of the mouth opening. The elastic element may be manufactured from a bio-compatible material. The elastic element may be manufactured from a range of materials, selected from latex, artificial rubbers, natural or synthetic rubbers, thermoplastic polymers, polystyrene, silicone and acetyl polymers or any other thermoplastic elastomer material.

According to other aspects of the invention, the elastic element may be inserted by the user into the mouth opening via the applicator. The applicator may include at least one handle which in turn is connected to at least two arms, wherein each arm may have at least one anchoring part configured to assist in holding one end of the restraining device while the restraining device is inserted into the mouth opening of the user and wherein the handle and the arms are one unit. The anchoring part may be selected from one of, a groove, a channel, a furrow, a rut, an indentation, a rough area and combinations thereof. The applicator may have release arms. Each of the release arms may have a limb which is at least one of, connected to and is a part of the release arm. The applicator may be made of stiff, but flexible material. The applicator may be one unit or may be a plurality of units.

According to another aspect of the invention, a method of inserting an intra-oral device into a user's mouth is envisioned, the method comprising biting an elastic restraining device until the restraining device enters between four teeth by force of the biting so that the device is located between two adjacent teeth in upper and lower jaws of a user, releasing pressure on an applicator configured to insert the device into a mouth opening of the user, thus freeing the restraining device from the applicator, and removing the applicator, without the restraining device, from the mouth opening of the user.

According to other aspects of the invention, the method may further comprise pulling and removing the restraining device from the mouth opening of the user. The restraining device may be used to be instructional as a teaching instrument for alteration of eating habits of the user.

According to yet another aspect of the invention, an applicator for inserting an elastic restraining device into a mouth cavity of a user comprises a central longitudinal handle having a holding end, a midsection, and a far end, a pair of near arms extending outwardly in opposite directions from the midsection of the handle, each arm having one end formed at the midsection of the handle and also having an outer end with a groove configured to hold an opposite end of the elastic restraining device, a pair of far arms extending outwardly in opposite directions from the far end of the handle and being configured to stretch the elastic restraining device, and a pair of release arms formed at one end at the outer ends of the near arms and extending rearwardly substantially parallel to the handle, wherein each of the release arms has an exterior side surface with castellations configured to aid the user in gripping the release arms.

According to other aspects of the invention, the pair of far arms may be arranged substantially perpendicular to the handle. The applicator may further comprise a pair of thin flexible limbs configured to connect the pair of release arms to the midsection of the handle. The pair of thin flexible limbs may be arranged substantially parallel to the pair of near arms. A pair of special grooves may be arranged one on each side of the holding end of the handle and configured to aid the user in removing the restraining device from the mouth cavity.

According to a further aspect of the invention, a system comprises a restraining device anchored in upper and lower jaws, including an elastic element which is inserted between two respective inter-proximal spaces of the upper and lower jaws. The elastic element includes a pair of inter-dental connectors, a slow-down chewing ring, and a pair of stoppers. The pair of connectors, the ring, and the pair of stoppers are connected together. An applicator is configured to insert the restraining device into a mouth opening of a user. The applicator includes a central longitudinal handle having a holding end, a midsection, and a far end. Special grooves are arranged on each side of the holding end of the handle and configured to aid the user in removing the restraining device from the mouth opening. The applicator includes two near arms, two release arms, and two far arms. Each near arm extends outwardly in opposite directions from the midsection of the handle. Each near arm has one end formed at the midsection of the handle and also has an outer end with at least one anchoring groove configured to assist in holding one end of the restraining device while the restraining device is inserted into the mouth opening of the user. Each of the release arms is formed at one end at the outer end of the near arms and extends rearwardly substantially parallel to the handle. Each of the release arms has a thin, flexible limb configured to connect each of the release arms to the midsection of the handle. Each thin, flexible limb is arranged substantially parallel to each of the near arms. Each of the release arms has an exterior side surface with castellations configured to aid the user in gripping the release arms. Each of the far arms is arranged substantially perpendicular to the handle. Each of the far arms extends outwardly in opposite directions from the far end of the handle and is configured to stretch the elastic restraining device. The handle, the near arms, the far arms, and the release arms comprise a single unit.

Other exemplary embodiments and advantages of the present invention may be ascertained by reviewing the present disclosure and the accompanying drawings.

### BRIEF DESCRIPTION OF THE DRAWINGS

The present invention is further described in the detailed description which follows, in reference to the noted plurality of drawings by way of non-limiting examples of embodiments of the present invention, in which like reference numerals represent similar parts throughout the several views of the drawings, and wherein:

FIG. 1 is a cross-sectional view of the oral environment in which the restraining device, i.e. the elastic element, is inserted;

FIG. 2 is a perspective view of the restraining device;

FIG. 3 is a side view of a first embodiment of the applicator holding the restraining device; and

FIG. 4 is a side view of a second embodiment of the applicator holding the restraining device.

### DETAILED DESCRIPTION OF THE PRESENT INVENTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

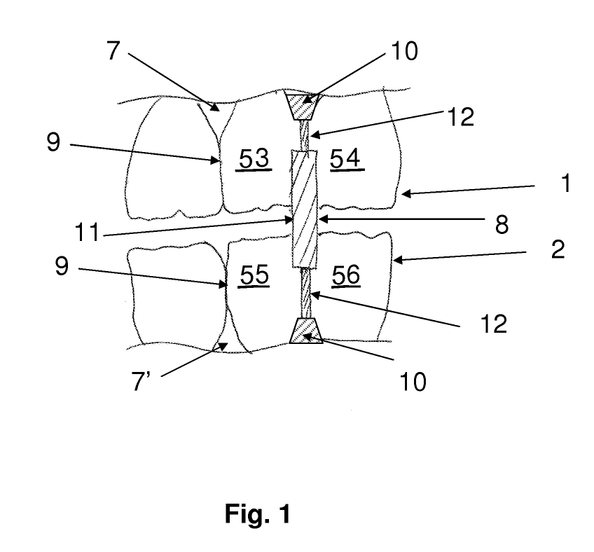

The system of the present invention includes a restraining device 8. FIG. 1 shows in cross section the oral environment in which the device 8 is applied. Teeth 53 and 54 in an upper jaw 1 (maxilla) in a mouth are disposed in opposition to teeth 55 and 56 in a lower jaw 2 (mandible). In each jaw 1 and 2, each tooth 53-56 typically forms a single contact point 9 with an adjacent tooth on each side, thus allowing for an approximately triangular space limited by adjacent teeth 53-56 and gums. Between adjacent upper and lower teeth, two inter-proximal spaces (IPSs) are indicated, namely IPS 7 and 7′, respectively. The restraining device 8, namely the elastic element as applied, is shown in cross section. When the device 8 is inserted between the two adjacent teeth 53 and 54 in the upper jaw 1 and between the two adjacent teeth 55 and 56 in the lower jaw 2, two elastic stoppers 10 at opposite ends of a slow-down chewing ring 11 are secured in the IPSs 7 and 7′ in order to retain the device 8 between the teeth 53-56. Thin inter-dental connectors 12 between the teeth 53-56 connect the stoppers 10 to the ring 11.

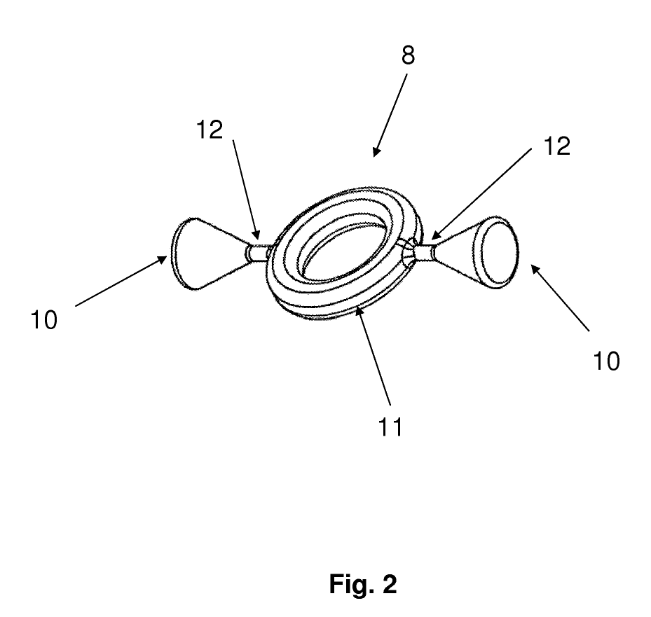

In FIG. 2, the device 8 is shown schematically with the two elastic stoppers 10 which are conical in shape and triangular in cross section. The stoppers 10 are secured at opposite sides of the slow-down chewing ring 11 by the inter-dental connectors 12.

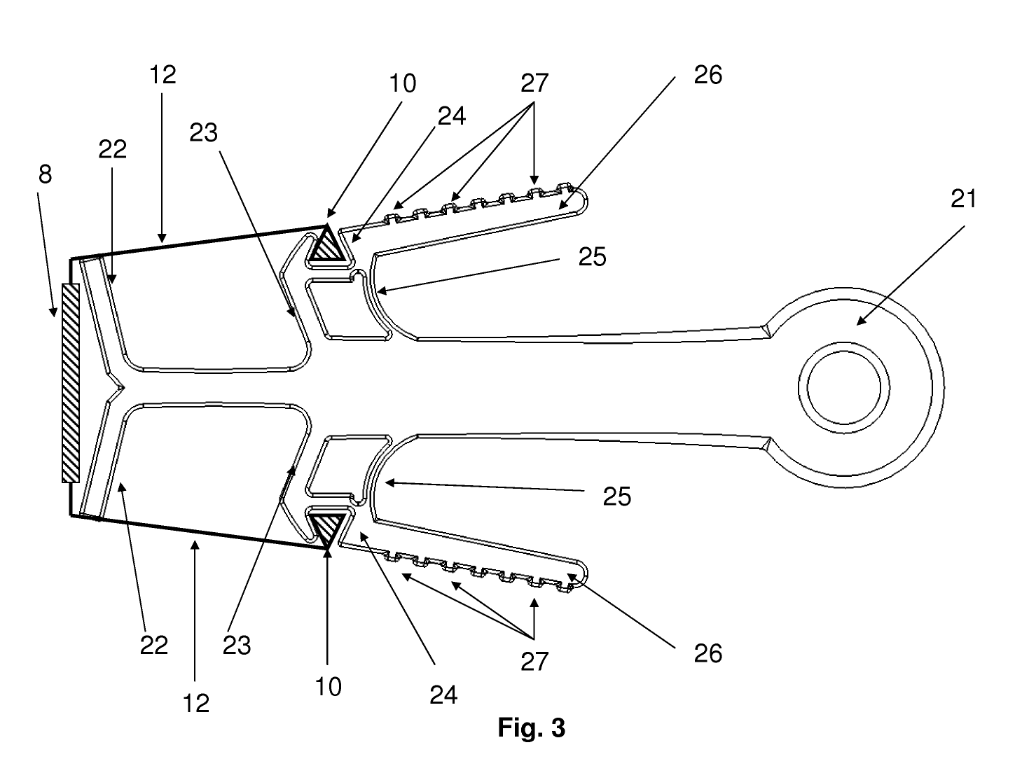

FIG. 3 shows a first embodiment of the applicator before use with the device 8 held thereon by stretching the connectors 12. The stoppers 10 at opposite ends of the connectors 12 are secured in grooves 24 of the applicator. The applicator has only one central handle 21 which is not meant to operate the device 8 but only to hold it. The handle 21 is connected to far arms 22 and near arms 23 which extend approximately perpendicular to a central longitudinal axis of the handle 21. After the user bites down on the stretched device 8, it is inserted between the teeth 53-56, as shown in FIG. 1. After the device 8 is inserted between the teeth 53-56, the user presses on release arms 26 which extend rearwardly at a slight angle from the central longitudinal axis of the handle 21, thus releasing the device 8 from the applicator. The release arms 26 are substantially parallel to the central longitudinal axis of the handle 21 and are able to be moved towards each other and the handle 21 between them because thin flexible limbs 25, which function as spring elements, bend and yield when the user presses upon exterior side surfaces of the release arms 26. Castellations 27 on the exterior side surfaces of the release arms 26 aid the user in gripping the release arms 26 securely without slippage. Now it is possible for the user to remove the applicator from the mouth while the device 8 remains firmly fitted between the upper jaw 1 and the lower jaw 2 in FIG. 1.

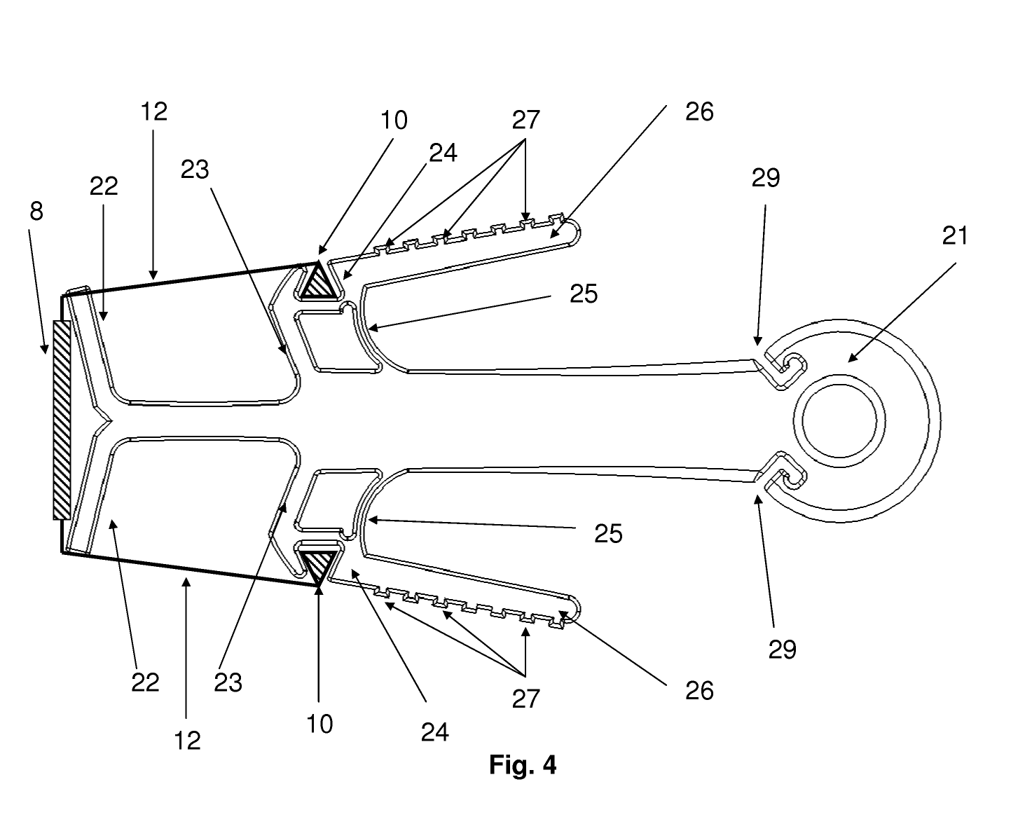

FIG. 4 shows a second embodiment of the applicator with the handle 21 having two special grooves 29 to assist the user in removing the device 8, after a meal, from its location in the mouth. The grooves 29 hook inside the ring 11 and allow the user to pull the device 8 from the mouth.

The device 8, as shown in FIG. 2, may be either a totally open or a partially closed structure. Moreover, it may have varied and different sizes, lengths, and thicknesses. It may also have different shapes, such as squares, rectangles, ellipses, polygons, etc., besides the circular ring shape shown in FIG. 2.

However, in accordance with the embodiment shown in FIG. 2, the device 8 has five parts: the one slow-down chewing ring 11, the two stoppers 10, and the two inter-dental connectors 12 which secure the stoppers 10 to opposite sides of the ring 11. Thus, the five parts 10-12 are connected to each other and comprise the one single compound elastic device 8.

The inter-dental connectors 12 are inserted between two adjoining teeth 53 and 54 or 55 and 56, into the IPSs 7 and 7′, as shown in FIG. 1. Hence the connectors 12 are flattened in order to pass between the contact points 9 of the adjoining teeth. The thickness of the connectors 12 may vary in accordance with the requirement for the user's teeth. However, in one embodiment, the thickness of the connectors 12 may be at least 0.5 mm. to as much as five mm. Besides the cylindrical shape shown in FIG. 2, the connectors 12 may be cut in the form of a circle, a rectangle, an ellipse, or the like. The connectors 12 may be made of rubber, silicone, nylon, or any other thermoplastic elastomer (TPE) material and manufactured as one component of the device 8.

The inter-dental connectors 12 must be flexible. Each stopper 10 is used for anchoring the device 8 in order to prevent it from being dislocated either from between the teeth 53-56 or from the connectors 12. Each stopper 10 is preferably located at opposite ends of the device 8, as shown in FIG. 2. Each stopper 10 is shown with a conical shape, but its geometrical shape may be triangular, circular, ring-like, disk-shaped (like a button), trapezoidal, rectangular, square, elliptical, etc.

While chewing, the device 8 is stretched, at which time each stopper 10 is pulled in such a way as to trap the restraining device 8 behind the teeth. Thus, this action impedes the restraining device 8 from coming free in the user's mouth. The diameter of each stopper 10 may vary from at least 0.5 mm to a maximum of five mm, depending upon the size of the user's mouth.

The slow-down chewing ring 11 is a flexible part which is stretched between the upper jaw 1 and the lower jaw 2 when opening the mouth. The dimensions of the ring 11 must be at least 10 mm but no more than 40 mm in its unstretched and relaxed position. The slow-down chewing ring 11 may have the cross-sectional form of a circle, as shown in FIG. 2, a rectangle or any other form, either hollow or solid.

The device 8 may be further formed into a variety of different shapes. The device 8 should be manufactured from a material which fulfills at least the following two conditions: first, a strong material which cannot be torn while being inserted into the mouth; and second, a flexible material which enables the opening and closing of the mouth. In one embodiment, this material is bio-compatible.

The device 8 may be manufactured from a range of TPE materials, such as latex, artificial rubbers, natural or synthetic rubbers, thermoplastic polymers, polystyrene, silicone, acetyl polymers, etc. of different Shore hardness.

Also, the device 8 may vary in diameter in different parts in order to control slowing down the action of opening the mouth.

In accordance with the preferred embodiments, the device 8 is inserted into its intended place with a helping instrument, such as the applicators shown in FIGS. 3 and 4.

Each applicator has at least one handle 21 which in turn is connected to at least one pair of near arms 23. Each near arm 23 has at least one anchoring part for inserting one stopper 10 of the device 8. Each applicator also has at least one pair of release arms 26, wherein the handle 21 and the arms 23 and 26 may be formed as one unit.

The anchoring part may be any part which will anchor the device 8, such as grooves 24, a channel, a furrow, a rut, an indentation, a rough area, or combinations thereof.

In accordance with the second embodiment, as shown in FIG. 4, the applicator may also have the release arms 26 which are gripped by the user in order to pull the device 8 from his mouth after eating. In other words, the handle 21 of the applicator serves as a grasping means for the user and has the release arms 26 to pull the device 8 out of the user's mouth. The release arms 26 of the applicator respond to a pressing action by the user.

The handle 21 is made of stiff material which may be either thermoplastic or thermosetting. The handle 21 may be hollow or solid. On the handle 21, there may be parts which direct the user to a pressing point.

The near arms 23 and far arms 22 of the applicator are used for grasping and stretching the device 8. The arms 22 and 23 may be either straight or bent or formed in varied shapes. The shapes of the arms 22 and 23 enable them to bend and to part from one another so that the device 8 is stretched over two or more arms 22 and 23.

The arms 22, 23 and 26 of the applicator may further have the following characteristics. The arms 22, 23 and 26 may be made of stiff material, e.g. plastic or metal. Also, the arms 22, 23 and 26 may be of different lengths.

The arms 22, 23 and 26 may be of different distances from one other. Also, the arms 22, 23 and 26 may vary in thickness, i.e. the upper part may be thicker than the lower part and vice versa. Also, any cut in an upper arm may be made in various shapes, e.g. round, rectangular, square, elliptic, etc.

In the second embodiment, as shown in FIG. 4, between the arms 23 and 26, which are close to the handle 21, there is the anchoring part, such as the groove 24, in a different cut than in the arms 22 that are far from the handle 21. Thus, the shape of the groove 24 enables the arms 23 and 26 to bend and part from one another while the device 8 is being stretched.

The anchoring parts, such as the grooves 24 or rough areas (not shown) on the arms 23, are located at the end of the arms 23. These parts serve to anchor the device 8 in order to stretch it. The parts are found in different sizes and in different shapes, e.g. straight cut, trapezoidal cut, triangular cut, etc. As stated above, the role of the parts is to anchor the device 8 to the arms 22 and 23 in order to allow the device 8 to stretch.

The applicator allows the user to insert the device 8 between the teeth 53-56 in a comfortable and user-friendly way before eating a meal. After the device 8 is inserted in its designated place and the meal is finished, the applicator may thereafter be applied to the user's mouth in order to remove the device 8 easily.

The applicator is an instrument which has the following characteristics. First, it stores the device 8, preferably in a stretched position, e.g. in a package, before it is used. Second, it assists a person in inserting the device 8 into the mouth, and in positioning it where it should be.

In assisting the user to insert the device 8 into its place in the mouth cavity, the distance between the arms 22 and 23 in FIGS. 3 and 4 is at least five mm and no more.

The applicator is made of stiff material but has flexible attributes. Also, the applicator may be one unit or more than one unit.

The device 8 slightly restricts mandible movement and therefore increases the awareness of the user to reduce opening his mouth and to slow down his rate of chewing the food which is ingested. A particular aspect of the present invention is the anchoring of the device 8 on each of the jaws 1 and 2. Because of the placement of the device 8 directly or via the connectors 12 and the stoppers 10, as herein before defined in the IPSs 7 and 7′, the anchoring of each jaw 1 and 2 is associated directly with two adjacent teeth 53, 54 and 55, 56, respectively.

The device 8 is easily applied by the user with or without the intervention of any medical personnel. The user, who may wish to do so, may remove the device 8 or more than one device, since one or more may be worn simultaneously, at any time. Reapplication is easy and there is no limit to the number of applications which may be carried out. The comfort of the application is conducive to the tendency of a person to use the device 8. The use of the device 8, or the use of one or more devices 8, would limit bite size, promote slow chewing, and limit the absorption of food.

More particularly, the applicator and the device 8 may be used as a system in accordance with the following method steps. However, the use of the system is not restricted to this method.

A. The applicator comes with the device 8 expanded upon it;

B. The user grasps the handle 21 of the applicator and brings it close to the user's mouth;

C. The user inserts the applicator into his mouth cavity with the device 8 stretched thereon;

D. The user bites on the device 8 until the connectors 12 enter between the teeth 53-56 by the force of the bite; thus, the device 8 positions itself between the two adjacent teeth 53 and 54 in the upper jaw 1 and between the two adjacent teeth 55 and 56 in the lower jaw 2, just like dental floss;

E. The user presses the release arms 26 in order to disengage the device 8 from the applicator; and

F. The user removes the applicator, without the device 8 attached thereto, from the user's mouth cavity.

A method of the present invention may also be described as comprising the steps of:

A. biting the elastic restraining device 8 until the device 8 enters between four teeth 53-56 by force of the biting so that the device 8 is located between two adjacent teeth 53 and 54 or 55 and 56 in the upper jaw 1 and the lower jaw 2, respectively, of the user;

B. releasing pressure on the applicator configured to insert the device 8 into the mouth opening of the user, thus freeing the device 8 from the mouth opening of the user; and

C. removing the applicator, without the device 8, from the mouth opening of the user.

The method may further comprise the step of pulling and removing the device 8 from the mouth opening of the user.

The device 8 may also be removed by the following methods. However, removal is not restricted to these methods. First, after using the device 8, the patient, by using his fingers, removes the device 8 and pulls it from his mouth cavity. In a second method, the handle 21 of the applicator serves as the removal device for the device 8.

The system comprises the device 8 which is anchored in the upper jaw 1 and the lower jaw 2. The device 8 is inserted into two respective IPSs 7 and 7′ of the upper jaw 1 and the lower jaw 2, thus increasing the awareness of the user to reduce and/or limit the mouth opening and/or to slow down the rate of chewing. An applicator is configured to insert the device 8 into the mouth opening of the user.

The elastic element of the device 8 includes at least one inter-dental connector 12, at least one slow-down chewing ring 11, and at least one stopper 10. The connector 12, the ring 11 and the stopper 10 are connected together.

The elastic element of the device 8 is manufactured from a strong material which cannot be torn while being inserted into the mouth opening and a flexible material which enables opening and closing of the mouth opening. The elastic element may also be manufactured from bio-compatible material. Furthermore, the elastic material may be manufactured from a range of materials selected from latex, artificial rubbers, natural or synthetic rubbers, thermoplastic polymer, polystyrene, silicone and acetyl polymers or any other thermoplastic elastomer material.

The elastic element of the device 8 is inserted by the user into the mouth opening via the applicator. The applicator includes at one handle 21 which is in turn connected to at least two arms 23, each having at least one anchoring part, e.g. the groove 24, configured to assist in holding the device 8 while the device 8 is inserted into the mouth opening of the user. The handle 21 and the arms 23 may be one unit. The anchoring part is selected from the groove 24, a channel, a furrow, a rut, an indentation, a rough area and combinations thereof.

The applicator also has the release arms 26. Each of the release arms 26 has the limb 25 which is connected to and/or is a part of the release arm 26. The applicator is made of stiff material but is flexible. The applicator may be either one unit or more than one unit.

As described above, the device 8 promotes the restriction of food intake and therefore helps the prone individual to overcome fast eating and excessive food intake.

In the method of the invention, the device 8 is used to be instructional as a teaching instrument for alteration of eating habits by alerting the user to reduce and/or limit the mouth opening and/or to slow down the rate of chewing.

The applicator is an important feature of the invention. It is used for inserting the device 8 into the mouth cavity of the user. The applicator includes the central longitudinal handle 21 having a holding end, a midsection, and a far end. The pair of near arms 23 extends outwardly in opposite directions from the midsection of the handle 21. Each arm 23 has one end formed at the midsection of the handle 21 and also has an outer end with the groove 24 configured to hold an opposite end, which includes the stopper 10, of the device 8. A pair of far arms 22 extends outwardly in opposite directions from the far end of the handle 21 and is configured to stretch the device 8. The pair of release arms 26 is formed at one end of the outer ends of the near arms 23 and extends rearwardly substantially parallel to the handle 21. Each of the release arms 26 has an exterior side surface with the castellations 27 configured to aid the user in gripping the release arms 26. The pair of far arms 22 is arranged substantially perpendicular to the handle 21. The pair of thin flexible limbs 25 is configured to connect the pair of release arms 26 to the midsection of the handle 21. The pair of thin flexible limbs 25 is also arranged substantially parallel to the pair of near arms 23. The pair of special grooves 29 in FIG. 4 is arranged one on each side of the holding end of wherein each of the release arms has a thin, flexible limb; the handle 2 and is configured to aid the user in removing the device 8 from the mouth cavity.

Continuous use of the device 8 helps train the user to achieve a proper chewing rate and promotes satiety without overeating. The use of the device 8 promotes a direct solution to dietary problems and also promotes the prone individual to acquire proper eating habits. By applying the device 8, the user may determine the size of the bite in such a way that a smaller amount of food is consumed in a given period of time. As a result, the user eats less and loses weight.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A system comprising:
a restraining device, which is anchored in upper and lower jaws, including an elastic element which is inserted between two respective inter-proximal spaces (IPSs) of the upper and lower jaws, thus increasing awareness of a user to at least one of, (a) reduce a mouth opening, and (b) slow down a rate of chewing; and
an applicator configured to insert the restraining device into the mouth opening of the user.

2. The system according to claim 1, wherein the restraining device includes at least one inter-dental connector, at least one slow-down chewing ring, and at least one stopper; wherein the connector, the ring and the stopper are connected together.

3. The system according to claim 1, wherein the elastic element is manufactured from a strong material which cannot be torn while being inserted into the mouth opening and a flexible material which enables opening and closing of the mouth opening.

4. The system according to claim 1, wherein the elastic element is manufactured from a bio-compatible material.

5. The system according to claim 1, wherein the elastic element is manufactured from a range of materials, selected from latex, artificial rubbers, natural or synthetic rubbers, thermoplastic polymers, polystyrene, silicone and acetyl polymers or any other thermoplastic elastomer material.

6. The system according to claim 1, wherein the elastic element is inserted by the user into the mouth opening via the applicator.

7. The system according to claim 6, wherein the applicator includes at least one handle which in turn is connected to at least two arms;
wherein each arm has at least one anchoring part configured to assist in holding one end of the restraining device while the restraining device is inserted into the mouth opening of the user; and
wherein said handle and said arms are one unit.

8. The system according to claim 7, wherein the anchoring part is selected from one of, a groove, a channel, a furrow, a rut, an indentation, a rough area and combinations thereof.

9. The system according to claim 7, wherein the applicator has release arms.

10. The system according to claim 9, wherein each of the release arms has a limb which is at least one of, connected to and is a part of the release arm.

11. The system according to claim 7, wherein the applicator is made of stiff material but is flexible.

12. The system according to claim 7, wherein the applicator is at least one unit.

13. The system according to claim 7, wherein the applicator is a plurality of units.

14. A method of inserting an intra-oral device into a user's mouth, said method comprising:
biting an elastic restraining device until the restraining device enters between four teeth by force of the biting so that the device is located between two adjacent teeth in upper and lower jaws of a user;
releasing pressure on an applicator configured to insert the device into a mouth opening of the user, thus freeing the restraining device from the applicator; and
removing the applicator, without the restraining device, from the mouth opening of the user.

15. The method according to claim 14, further comprising:
pulling and removing the restraining device from the mouth opening of the user.

16. The method according to claim 14, further comprising:
using the restraining device to be instructional as a teaching instrument for alteration of eating habits of the user.

17. An applicator for inserting an elastic restraining device into a mouth cavity of a user, said applicator comprising:
a central longitudinal handle having a holding end, a midsection, and a far end;
a pair of near arms extending outwardly in opposite directions from the midsection of the handle, each arm having one end formed at the midsection of the handle and also having an outer end with a groove configured to hold an opposite end of the elastic restraining device;
a pair of far arms extending outwardly in opposite directions from the far end of the handle and being configured to stretch the elastic restraining device; and
a pair of release arms formed at one end at the outer ends of the near arms and extending rearwardly substantially parallel to the handle, wherein each of the release arms has an exterior side surface with castellations configured to aid the user in gripping the release arms.

18. The applicator according to claim 17, wherein the pair of far arms is arranged substantially perpendicular to the handle.

19. The applicator according to claim 17, further comprising:
a pair of thin flexible limbs configured to connect the pair of release arms to the midsection of the handle.

20. The applicator according to claim 19, wherein the pair of thin flexible limbs is arranged substantially parallel to the pair of near arms.

21. The applicator according to claim 19, further comprising:
a pair of special grooves arranged one on each side of the holding end of the handle and configured to aid the user in removing the restraining device from the mouth cavity.

22. A system comprising:
a restraining device anchored in upper and lower jaws, including an elastic element which is inserted between two respective inter-proximal spaces of the upper and lower jaws;
the elastic element including a pair of inter-dental connectors, a slow-down chewing ring, and a pair of stoppers;
said pair of connectors, said ring, and said pair of stoppers being connected together;
an applicator configured to insert the restraining device into a mouth opening of a user;
the applicator including a central longitudinal handle having a holding end, a midsection, and a far end;
special grooves arranged on each side of the holding end of the handle and configured to aid the user in removing the restraining device from the mouth opening;
said applicator further including two near arms, two release arms, and two far arms;
each near arm extending outwardly in opposite directions from the midsection of the handle;
each near arm having one end formed at the midsection of the handle and also having an outer end with at least one anchoring groove configured to assist in holding one end of the restraining device while the restraining device is inserted into the mouth opening of the user;
each of the release arms being formed at one end at the outer end of the near arms and extending rearwardly substantially parallel to the handle;
each of the release arms having a thin, flexible limb configured to connect each of the release arms to the midsection of the handle;
each thin, flexible limb being arranged substantially parallel to each of the near arms;
each of the release arms having an exterior side surface with castellations configured to aid the user in gripping the release arms;
each of the far arms being arranged substantially perpendicular to the handle;
each of the far arms extending outwardly in opposite directions from the far end of the handle and being configured to stretch the elastic restraining device; and
said handle, said near arms, said far arms, and said release arms comprising a single unit.
